SEQUENCE LISTING (1 sequence)

SEQ ID NO: 1 - DNA, 37 bp, synthetic construct
  source 1..37: mol_type = other DNA
tttttttcactcattcaataccctacgtctaccctac

(19) United States
(12) Patent Application Publication
(10) Pub. No.: US 2024/0280585 A1
(43) Pub. Date: Aug. 22, 2024

(54) METHODS OF MEASURING PROTEIN LEVELS OF CELLS AND USES THEREOF

(71) Applicant: CITY UNIVERSITY OF HONG KONG, Kowloon (HK)

(72) Inventors: Chia-Hung CHEN, Kowloon (HK); Ying XU, Kowloon (HK)

(21) Appl. No.: 18/443,282

(22) Filed: Feb. 15, 2024

Related U.S. Application Data
(60) Provisional application No. 63/485,535, filed on Feb. 16, 2023.

(51) Int. Cl. G01N 33/68; G01N 33/50; G01N 33/58

(52) U.S. Cl. CPC G01N 33/6854; G01N 33/5047; G01N 33/582

(57) ABSTRACT

Disclosed herein is a method of measuring the level of at least one protein of a cell. According to some embodiments of the present disclosure, the method comprises, mixing the cell with a cholesterol-linked antibody (CLAb) specific to a protein secreted by the cell, encapsulating the mixing product in a water-in-oil droplet for a period of time, extracting the cells from the droplet, and labeling the cells with a fluorophore-linked antibody (FLAb) so as to measure the level of the protein. Also disclosed herein is a method of treating a cancer in a subject via measuring the protein levels of a biological sample of the subject with the aid of the present measurement method and subjecting the subject in need thereof an anti-cancer treatment.

### CROSS-REFERENCE TO RELATED APPLICATIONS

This application relates to and claims the benefit of U.S. Provisional Application No. 63/485,535 filed Feb. 16, 2023 in the United States Patent and Trademark Office; the content of the application is incorporated herein by reference in its entirety.

### SEQUENCE LISTING XML

The present application is being filed along with a Sequence Listing XML in electronic format. The Sequence Listing XML is provided as an XML file entitled HPO291US_SEQ_AF, created Dec. 15, 2023, which is 2 Kb in size. The information in the electronic format of the Sequence Listing XML is incorporated herein by reference in its entirety.

### BACKGROUND OF THE INVENTION

### 1. Field of the Invention

The present disclosure in general relates to the field of protein measurement. More particularly, the present disclosure relates to a method for measuring protein levels of single cells by using cholesterol-linked antibodies (CLAbs) with the aid of droplet technology.

### 2. Description of Related Art

Cellular secretions typically function as mediators of bio-signal transduction and thus play an important role in a range of physiological and pathological processes. For example, the levels of interferon-gamma (IFN-γ), interleukin (IL)-1b, IL-6, monocyte chemoattractant protein-1 (MCP-1), tumor necrosis factor-alpha (TNF-α), IL-2, and IL-5 are significantly elevated in the plasma of nasopharyngeal carcinoma (NPC) patients, subsequently undergo a marked reduction following treatment. Notably, as immune cells exhibit functional diversity, even the genetically homogeneous cells can demonstrate a range of different physiological functions, due to the cell heterogeneous response to the varied clinical conditions. It is necessary to measure single-cell cytokines or chemokines, which can yield more comprehensive understanding of immune response and facilitate diagnosis of diseases or prognostic evaluation of therapeutic efficacy. By screening secreted single cells in patients, critical cells with active immune expressions could be isolated and profiled to guide therapeutic strategies in clinical practice. However, there are currently no effective assays for use at scale.

In view of the foregoing, there is a continuing interest in developing a novel method for measuring protein levels of single cells.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a method of measuring the level of at least one protein of a cell. According to the embodiments of the present disclosure, the method comprises,
(a) mixing the cell with a cholesterol-linked antibody (CLAb) to produce a CLAb-conjugated cell, wherein the CLAb comprises a first antibody specific to a first protein secreted from the cell, and a cholesterol linked to the first antibody;
(b) encapsulating the CLAb-conjugated cell of step (a) in a water-in-oil droplet;
(c) incubating the product of step (b) at a temperature for a period of time;
(d) adding a de-emulsifier to the product of step (c);
(e) mixing the product of step (d) with a solution comprising a first fluorophore-linked antibody (FLAb), wherein the first FLAb comprises a second antibody also specific to the first protein secreted from the cell, and a first fluorophore linked to the second antibody;
(f) irradiating the product of step (e) with a light having a first wavelength to excite the first fluorophore; and
(g) detecting the intensity of a first signal emitted by the excited first fluorophore of step (f) at a second wavelength, in which the first signal represents the measurement of the level of the first protein.

According to certain preferred embodiments of the present disclosure, in step (c), the product of step (b) is incubated at 37° C. for at least 4 hours. In one exemplary embodiment, the product of step (b) is incubated at 37° C. for 6 hours.

Optionally, the solution of step (e) further comprises a second FLAb, which comprises a third antibody specific to a second protein expressed on the surface of the cell, and a second fluorophore linked to the third antibody, wherein the first and second fluorophores are different. In this case, the method further comprises the steps of,
(f-1) irradiating the product of step (e) with a light having a third wavelength to excite the second fluorophore before or concurrent with step (f); and
(g-1) detecting the intensity of a second signal emitted by the excited second fluorophore of step (f-1) at a fourth wavelength, in which the second signal represents the measurement of the level of the second protein.

Optionally, in step (b), the CLAb-conjugated cell of step (a) is encapsulated with a cell stimulant in the water-in-oil droplet. According to certain exemplary embodiments, the cell is an immune cell, and the cell stimulant is concanavalin A (Con A), phorbol myristate acetate (PMA), phytohaemagglutinin (PHA), ionomycin (Iono), lipopolysaccharide (LPS), or a combination thereof.

Also disclosed herein is a method of treating a cancer in a subject. The method comprises,
(a) isolating a biological sample from the subject;
(b) measuring the level of at least one protein in the biological sample of step (a) by the measurement method as described above;
(c) comparing the level of the at least one protein measured in step (b) with that in a control sample derived from a healthy subject; and
(d) subjecting the subject to an anti-cancer treatment based on the result of step (c), wherein the level of the at least one protein in the biological sample of the subject is higher or lower than that in the control sample.

According to some embodiments, the anti-cancer treatment is surgery, chemotherapy, radiation therapy, immunotherapy, hormone therapy, anti-angiogenic therapy, or a combination thereof.

According to some exemplary embodiments, the biological sample is a peripheral blood mononuclear cell (PBMC) sample. In the embodiments, the protein is IFN-γ, TNF-α, tumor growth factor-beta (TGF-β), IL-1, IL-4, IL-5, IL-6, IL-8, IL-10, IL-13, IL-16, IL-17, IL-23, MCP-1, or a combination thereof. According to one specific example, the cancer is nasopharyngeal carcinoma (NPC), and the level of the IFN-γ and TNF-α in the biological sample of the subject is higher than that in the control sample.

In all embodiments of the present disclosure, the subject is a mammal; preferably, a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings briefly discussed below.

FIG. 1 shows schematic of the cell membrane immunosorbent assay based on cholesterol-linked antibody (CLAb) technology. There are two steps for the single-cell screen of secretions and surface proteins: Step 1. Capture secreted proteins with CLAbs on the cell surface in droplets; and Step 2. Label the captured proteins with fluorophore-tagged antibodies in bulk. The single cells with barcoded antibodies were then screened via flow cytometry for multiplexed analysis.

FIGS. 2A-2I demonstrates CLAb synthesis and characterization. (a) Cholesterol-linked DNA strands and DBCO-modified antibodies were conjugated via a copper-free click chemistry method to fabricate CLAbs. (b) SDS-PAGE was conducted to characterize the synthesized CLAbs. Marker: Protein ladder, Control: Unmodified anti-IL-16 antibodies, and CLAb: CLAb-IL-16. (c) 3T3, THP-1 and Raji cells grafted with CLAbs labeled by FITC-conjugated goat anti-mouse IgG were observed under a microscope to record bright-field and fluorescent images. (d) The stability of CLAbs (CLAb-IL-16) on the Raji cell surface was monitored by recording FITC fluorescence signals for 24 hrs. (e) The bar chart for the FITC fluorescence change (from 596.5±282.14 a.u. to 1355±134.35 a.u., n=3, mean±s.d.) with different CLAb-cell ratios (from 1.6×106:1 to 8×106:1) was used to optimize the experimental conditions. (f) A flow cytometry assay evaluated Raji cells grafted with CLAb-IFN-γ to capture IFN-γ at various concentrations. (g) The CLAb-IFN-γ capture efficiency was calibrated by measuring MFI changes (from 26.53±1.44 a.u. to 5712.33±960.13 a.u., n=3, mean±s.d.) resulting from increases in IFN-γ concentrations (from 0 pg/mL to 105 pg/mL). (h) High specificity for the measurement of target cytokines by CLAbs (CLAb-IFN-γ, CLAb-TNF-α and CLAb-IL-16) was exhibited, as shown in the heatmap. (i) A multiplexed assay for IFN-γ (5×104 pg/mL) and TNF-α (2×104 pg/mL) was performed by simultaneously grafting CLAb-IFN-γ and CLAb-TNF-α on the same cells, and the results are shown as flow cytometry pseudocolor plots.

FIGS. 3A-3H represents single-cell multiplexed assay for multiple cytokines and surface proteins. (a) Single-cell encapsulation in droplets (average: 20.57 pl, SEM: 1.76 pl) was observed under a microscope. (b) The single-cell encapsulation rate for the droplets was characterized (black line, n=25) and compared with the Poisson distribution (red line). (c) The high cell survival rate (˜99.5%) of THP-1 cells after extraction from the droplets through de-emulsification was determined by using live/dead stains. (d) The CLAb anchoring stability on the THP-1 cell surface after de-emulsification was characterized by evaluating FITC-conjugated goat anti-mouse IgG fluorescence signals. Most CLAbs (91.05%) remained on the cell surface when comparing the CLAb-grafted cells before and after de-emulsification. (e) Different expression levels of IFN-γ and TNF-α among secretory THP-1 cells, DCs and M1 were assessed by using an Ordinary one-way ANOVA test. P values: ns>0.05, * 0.05-0.01, ** 0.01-0.001, *** 0.001-0.0001, ****<0.0001. (f) UMAP/tSNE plot based on different clustering conditions. (g) By using FlowSOM facilitated with UMAP, 12 cell clusters were identified based on the expression levels of IFN-γ, TNF-α and CD13 to distinguish different cell types (THP-1, DC and M1). The fractions of different cell clusters within each cell type are displayed in a stacked chart, and the scaled total MFI of each marker (IFN-γ, TNF-α, and CD13) is displayed in a heatmap. (h) The cluster frequency is depicted in bar graphs.

FIGS. 4A-4F shows high-dimensional single-cell data clustering for clinical analysis. (a) Using FlowSOM facilitated with UMAP, 12 cell clusters were generated based on the expression of surface proteins (CD3, CD4, CD8 and CD19) and secretions (IFN-γ and TNF-α). (b) The fractions of each cluster under different states (basal, stimulations with 50 ng/mL PMA and 1 μg/mL ionomycin, 1.25 μg/mL Con A, and 1.25 μg/mL PHA-L) are displayed in a stacked chart. (c) A FlowSOM tree was built to show all clusters with marker expression levels. Clusters are displayed as circles with star plots showing median marker (IFN-γ, CD19, TNF-α, CD3, CD4 and CD8) intensities of the cluster. The 12 different background colors of circles represent 12 clusters. Legends of the star plot and clusters are shown on the right side. (d) The scaled total MFI of each marker (IFN-γ, CD19, TNF-α, CD3, CD4 and CD8) is displayed in the heatmap. (e) Cluster frequencies for each state (basal, stimulated by PMA/ionomycin, Con A or PHA-L) were plotted in bar graphs (N=1, cell number for each condition=3000). (f) The median fluorescence intensities of IFN-γ and TNF-α were quantified for all cells in each sample obtained from four healthy donors (HD) and four NPC patients (P) under different states (basal, stimulated by PMA/ionomycin) to reveal their distinct immune functions (cell number for each healthy donor/NPC patient=3000). Error bars show the mean standard deviation of median fluorescence intensities. T-test analysis of average IFN-γ and TNF-α secretion levels for all cells among NPC patients and healthy donors. Differences in IFN-γ and TNF-α secretion levels were assessed with two-tailed, unpaired t-tests with 95% confidence. P values: ns>0.05, * 0.05-0.01, ** 0.01-0.001, *** 0.001-0.0001, ****<0.0001. N=number of healthy donors/NPC patients, n=number of repeated assays for each donor. a.u.: arbitrary units.

FIG. 5 demonstrates the proportion of cells secreting IFN-γ and/or TNF-α among healthy donors (HD) and NPC patients (P) in basal state or under PMA/ionomycin stimulation (n=3, for the independent experiments, cell number for each condition of HD/P=3000). Error bars show mean standard deviations. Differences in percentage of secreted cells between healthy donors and NPC patients in the basal state were assessed with two-tailed, unpaired T-tests with 95% confidence (N=4, n=3 repeated assays for each donor, NPC patients 2.82±1.481%; healthy donors 0.70±0.29%, mean±s.d., P<0.0001). After PMA/ionomycin stimulation, more secreted cells were observed in the NPC patient samples compared with healthy donors (N=4, n=3 repeated assays for each donor, NPC patients 31.11±6.32%; healthy donors 23.01±11.06%, mean±s.d., P=0.038). P values: ns>0.05, * 0.05-0.01, ** 0.01-0.001, *** 0.001-0.0001, ****<0.0001.

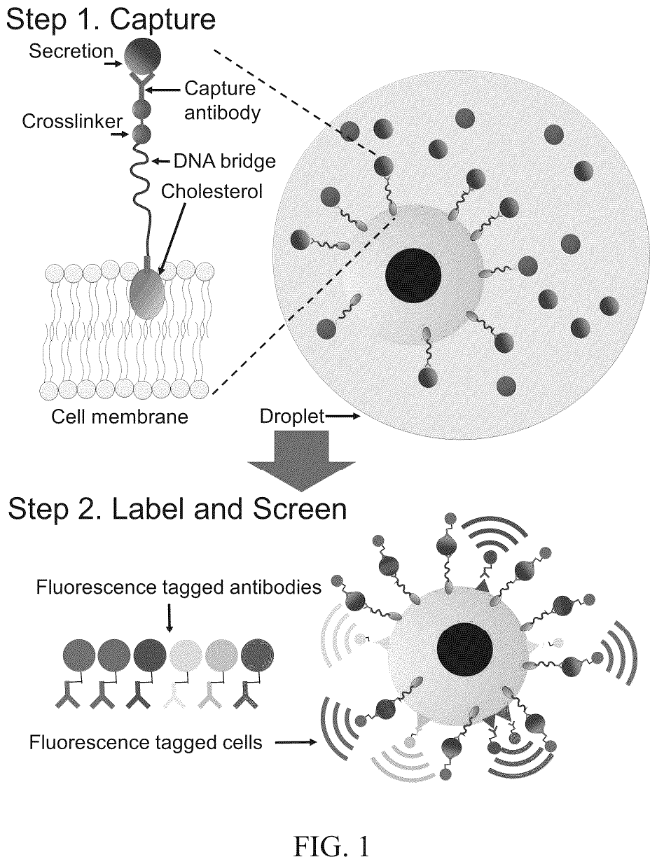

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

In the present specification and claims, the term “antibody” is used in the broadest sense and covers fully assembled antibodies, antibody fragments that bind with antigens, such as antigen-binding fragment (Fab/Fab′), F(ab′)₂ fragment (having two antigen-binding Fab portions linked together by disulfide bonds), variable fragment (Fv), single chain variable fragment (scFv), bi-specific single-chain variable fragment (bi-scFv), nanobodies, unibodies and diabodies. “Antibody fragments” comprise a portion of an intact antibody, preferably the antigen-binding region or variable region of the intact antibody. Typically, an “antibody” refers to a protein consisting of one or more polypeptides substantially encoded by immunoglobulin genes or fragments of immunoglobulin genes. The well-known immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. A typical immunoglobulin (antibody) structural unit is known to comprise a tetramer. Each tetramer is composed of two identical pairs of polypeptide chains, with each pair having one “light” chain (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

The term “water-in-oil” refers to an emulsion in which a more hydrophilic or apolar solution or suspension (e.g., a suspension containing the present CLAb-conjugated cell, and optionally, one or more cell stimulants) is encapsulated in a more hydrophobic or polar encapsulant phase (e.g., an carrier oil). The term “droplet” refers to a state of liquid ejected from a liquid jetting device, and may be a granular shape, a teardrop shape, or a tailing shape. The droplets referred to herein are composed of a material which can be ejected by the liquid jetting device. As used herein, the term “water-in-oil droplet” means a macroscopically homogeneous, kinetically stable droplet comprising at least two mutually immiscible phases; one being the dispersing continuous oily phase and the other being the aqueous phase dispersed in the said continuous oily phase in the form of droplets. The two phases are kinetically stabilized by at least one emulsifying system generally comprising at least one emulsifying surfactant.

The term “fluorophore” as used herein refers to a molecule that absorbs energy of a specific wavelength and re-emits energy at a different wavelength. For example, in the case when the fluorophore is FITC, it may be excited by a light having a wavelength of 485-495 nm (a first wavelength), and emits light at 520-530 nm (a second wavelength).

As used herein, the term “treat,” “treating” and “treatment” are interchangeable, and encompasses partially or completely preventing, ameliorating, mitigating and/or managing a symptom, a secondary disorder or a condition associated with cancer. The term “treating” as used herein refers to application or administration of one or more anti-cancer treatments to a subject, who has a symptom, a secondary disorder or a condition associated with cancer, with the purpose to partially or completely alleviate, ameliorate, relieve, delay onset of, inhibit progression of, reduce severity of, and/or reduce incidence of one or more symptoms, secondary disorders or features associated with the cancer. Symptoms, secondary disorders, and/or conditions associated with cancers include, but are not limited to, pain, bleeding, lump, weight loss, fever, fatigue, difficulty breathing, difficulty swallowing, and night sweat. Treatment may be administered to a subject who exhibits only early signs of such symptoms, disorder, and/or condition for the purpose of decreasing the risk of developing the symptoms, secondary disorders, and/or conditions associated with cancer. Treatment is generally “effective” if one or more symptoms or clinical markers are reduced as that term is defined herein. Alternatively, a treatment is “effective” if the progression of a symptom, disorder or condition is reduced or halted.

The term “subject” refers to a mammal including the human species that is treatable with methods of the present invention. The term “subject” is intended to refer to both the male and female gender unless one gender is specifically indicated.

The term “healthy subject” refers to a subject that does not have a disease (e.g., a cancer). For example, a healthy subject has not been diagnosed as having a disease and is not presenting with two or more (e.g., two, three, four or five) symptoms associated with the disease.

### II. Description of the Invention

The present invention aims at providing a method for simultaneously measuring the levels of a plurality of proteins (e.g., one or more secreted proteins and/or one or more surface proteins) of single cells. Since cellular secretion plays a key role in physiological and pathological process, in which the level of specific protein(s) may serve as marker(s) of a disease (e.g., cancer), a diagnosis of the disease may thus be rendered via measuring the level of that specific protein(s) secreted from individual cells in a biological sample obtained/derived from the subject by the present method. Once the subject is diagnosed with the disease, a suitable treatment (e.g., an anticancer treatment) may then be timely administered to the subject.

Accordingly, the first aspect of the present disclosure is directed to a method of measuring the level of secreted protein(s) of a cell. According to embodiments of the present disclosure, the method comprises steps of,
(a) mixing the cell with a first CLAb specific to a protein secreted from the cell (i.e., a secreted protein of the cell);
(b) encapsulating the product of step (a) in a water-in-oil droplet;
(c) incubating the product of step (b) at a temperature for a period of time;
(d) adding a de-emulsifier to the product of step (c);
(e) mixing the product of step (d) with a solution comprising a first fluorophore-linked secondary antibody (FLAb) specific to the secreted protein;
(f) irradiating the product of step (e) with a light having a first wavelength to excite the first FLAb; and
(g) detecting the intensity of a signal emitted by the excited first FLAb of step (f) at a second wavelength, in which the signal represents the measurement of the level of the secreted protein.

The present method commences by mixing a population of cells derived from the subject with a CLAb (i.e., a first CLAb). According to the embodiments of the present disclosure, the CLAb comprises, in its structure, a primary antibody specific to the secreted protein of the cell, and a cholesterol linked to the primary antibody. The cholesterol is capable of interacting with lipid components (e.g., phosphatidylcholines and/or sphingolipids) on the cell membrane thereby integrating itself into cell membrane via lipid-cholesterol interaction. The thus-produced “CLAb-conjugated cell” has the primary antibody anchored on its cell surface via the cholesterol of the CLAb. According to one exemplary embodiment, the CLAb is an anti-IFN-γ CLAb, which has a primary antibody specific to IFN-γ. According to another exemplary embodiment, the CLAb is an anti-TNF-α CLAb, which has a primary antibody specific to TNF-α.

Optionally, the CLAb further comprises a linker, e.g., a DNA linker or polyethylene glycol (PEG) linker, linking the cholesterol to the primary antibody. According to certain embodiments of the present disclosure, the cholesterol is linked to the primary antibody via a DNA linker. In these embodiments, the CLAb is produced by coupling a cholesterol-conjugated DNA linker to a dibenzocyclooctyne group (DBCO)-modified primary antibody. Specifically, the primary antibody is first modified with an adaptor DNCO-sulfo-NHS, which, as its name implies, has an N-hydroxysuccinimide (NHS) group at one end, and a DBCO group at the other end. The NHS group of the adaptor may react with the amine group on the side chain of the primary antibody, thereby forming the DBCO-modified primary antibody. In this case, a DNA linker having the cholesterol linked to one end and an azide group linked to the other end is capable of linking to the DBCO-modified primary antibody via a copper-free click reaction occurred between the azide and DBCO groups. As could be appreciated, the azide or DBCO group may be alternatively substituted by different groups suitable for click chemistry, for example, an alkyne, tetrazine, or trans-cyclooctyne (TCO) group.

In step (b), the CLAb-conjugated cell is encapsulated in a water-in-oil droplet. According to some embodiments of the present disclosure, the step (b) is carried out with the aid of a microfluidic droplet generator, which includes one inlet for the continuous oil phase, and one inlet for the aqueous phase. The microfluidic droplet generator is useful in loading the cell suspended in the aqueous phase into the carrier oil (for example, surfactant-rich biocompatible emulsion, e.g., propylene glycol tert-octylphenyl ether or sorbitan monolaurate; or biocompatible oil, e.g., isopropyl palmitate and caprylic/capric/linoleic triglyceride). The method for encapsulating a molecule (e.g., the present CLAb-conjugated cell) in a carrier oil is known in the art; hence, for the sake of brevity, the detailed description is omitted herein.

Optionally, the CLAb-conjugated cell is encapsulated with one or more cell stimulants in the carrier oil. To this purpose, the microfluidic droplet generator is employed to produce the water-in-oil droplet. The microfluidic droplet generator may include one inlet for the continuous oil phase, and two inlets for the aqueous phase respectively for loading the present CLAb-conjugated cell and cell stimulant(s). Depending on intended purpose, the cell stimulant may be any agents known to stimulate the proliferation, differentiation, activation, protein expression, and/or protein secretion of a cell. According to certain embodiments of the present disclosure, the cell is an immune cell, in which the cell stimulant may be Con A, PMA, PHA, Iono, LPS, TT, or a combination thereof. In one specific example, the cell is PBMC, and the cell stimulant is the combination of PMA and Iono.

In step (c), the product of step (b) (i.e., the water-in-oil droplet containing CLAb-conjugated cell with or without cell stimulant(s)) is incubated at a suitable temperature for a sufficient period time so that the protein secreted from the cell and encapsulated in the water-in-oil droplet may be captured by the membrane-anchored CLAb, i.e., in the form of an “secreted protein-CLAb-cell immunoconjugate”. According to some preferred embodiments of the present disclosure, the product of step (b) is incubated at 37° C. for at least 4 hours, such as 4, 5, 6, 7, 8, 9, 10, 11 or 12 hours, or longer. In one example, the product of step (b) is incubated at 37° C. for 6 hours. A skilled artisan may adjust the incubation condition (including the temperature and time) in accordance with practical needs.

In step (d), a de-emulsifier is added to the product of step (c) to release the secreted protein-CLAb-cell immunoconjugate from the carrier oil. As known to a skilled artisan in the art, a de-emulsifier refers to an agent that neutralizes the effects of an emulsifier via altering the ionic character of the emulsifier and de-stabilizing the slurry into an aqueous phase and a microcapsule rich phase. Examples of de-emulsifier suitable to be used in the present method include, but are not limited to, metal salt (e.g., sodium, aluminum, calcium, or ferric salt), non-metal salt (e.g., borate, calcium chloride), polymer (e.g., cationic polymer, anionic polymer, non-ionic polymer, cationic polyamine, cationic polyacrylamide, etc.), and ethanol. According to some exemplary embodiments, the de-emulsifier is a fluorocarbon-based polymer. In one specific example, the de-emulsifier is a fluorotelomer alcohol (FTOH; a fluorotelomer with an alcohol functional group).

Then, in step (e), the product of step (d) is mixed with a solution containing a FLAb (i.e., a first FLAb) specific to the secreted protein. The FLAb comprises, in its structure, a secondary antibody specific to the secreted protein with different epitopes from the primary antibody of the CLAb, and a fluorophore linked to the secondary antibody. Accordingly, the FLAb is capable of binding to the CLAb-conjugated cell via interacting with the secreted protein, and forming a FLAb-secretion-CLAb-conjugated cell.

The solution containing the FLAb may be any staining buffer known in the art that minimizes non-specific binding of antibody and antigen, for example, a phosphate-buffered saline (PBS) containing fetal calf serum (FCS, e.g., 1-10% FCS), bovine serum albumin (BSA; e.g., 0.5-1% BSA), and/or fetal bovine serum (FBS; e.g., 1-10% FBS), in the absence or presence of ethylenediaminetetraacetic acid (EDTA) and/or sodium azide.

Depending on intended purpose, the fluorophore may be any molecule that absorbs energy of a specific wavelength and re-emits energy at a different wavelength. Exemplary fluorophores suitable for use in the present invention include, but are not limited to, cyan fluorescent protein (CFP), green fluorescent protein (GFP), yellow fluorescent protein (CFP), red fluorescent protein (CFP), far-red fluorescent protein (FFP), infrared fluorescent protein (IFP), fluorescein isothiocyanate (FITC), phycoerythrin (PE), allophycocyanin (APC), carboxy-X-rhodamine (ROX), Texas red, cyanine dye (for example, Cy2, Cy3, Cy5 or Cy7), and Alexa Fluor® dye (e.g., Alexa Fluor®488, Alexa Fluor®430, Alexa Fluor®532, Alexa Fluor®546, Alexa Fluor®555, Alexa Fluor®568, Alexa Fluor®594, Alexa Fluor®644, Alexa Fluor®660 or Alexa Fluor®680).

Next, the FLAb-protein-CLAb-conjugated cell of step (e) is irradiated with a light having a first wavelength to excite the fluorophore of the first FLAb (step (f)), and then the signal emitted from the excited fluorophore at a second wavelength is detected, in which the signal represents the measurement of the level of the secreted protein (step (g)). As would be appreciated, the first and second wavelengths vary with the fluorophore selected. A skilled artisan may choose suitable first and second wavelengths in accordance with the general knowledge in the art (i.e., the excitation/absorption and emission spectrums corresponding to the intended fluorophores).

According to some embodiments of the present disclosure, the steps (f) and (g) are carried out by use of a flow cytometry, a technique known in the art that can determine whether the protein of interest is expressed by a cell, as well as the amount of the protein expressed by the cell (i.e., quantifying the protein expression level) based on the fluorescence intensity. In general, samples are analyzed in a flow cytometer to produce an array of information about cell. Detectors aimed directly in line with a single laser beam (forward scatter, FSC) determine cell size while detectors aimed perpendicular to the laser beam (side scatter, SSC) assess granularity within the cytoplasm of cells. Fluorescent detectors within the flow cytometer are used to determine fluorescence intensity emitted from cells, which indicates the level of the protein of interest. Preferably, approximately 12 fluorophores may be analyzed at one time via employing different fluorescent detectors.

Accordingly, the present method is useful in measuring the levels of more than one secreted proteins (for example, two, three, four, five, six, or more secreted proteins) at one time, in which the cells are mixed with different CLAbs (e.g., a first, second, third, fourth, fifth and sixth CLAbs respectively specific to the first, second, third, fourth, fifth and sixth secreted proteins) and FLAbs (e.g., a first, second, third, fourth, fifth and sixth FLAbs respectively specific to the first, second, third, fourth, fifth and sixth secreted proteins) in steps (a) and (e), and then respectively irradiating/exciting the FLAbs and detecting the energies emitted therefrom at the corresponding wavelengths of each fluorophores of the FLAbs in steps (f) and (g). Specifically, the steps of the method for quantifying multiple secreted proteins are quite similar to that of the method for quantifying one secreted protein as described above, except the followings:
(i) The cells are mixed with more than one CLAbs in step (a), in which the CLAbs respectively comprise primary antibodies specific to the secreted proteins intended to be measured; for example, in the case when the method is used to measure respective levels of IFN-γ and TNF-α, then two CLAbs (an anti-IFN-γ CLAb and an anti-TNF-α CLAb) are mixed with the cells so as to respectively capture the IFN-γ and TNF-α secreted from the cell; alternatively, in the case when the method is used to measure respective levels of IFN-γ, TNF-α and IL-16, then three CLAbs (an anti-IFN-γ CLAb, an anti-TNF-α CLAb and an anti-IL-16 CLAb) are mixed to the cells so as to respectively capture the IFN-γ, TNF-α and IL-16 secreted from the cell;
(ii) Each secreted protein is also recognized and bound by a corresponding FLAb carrying a specific fluorophore in step (e); for example, in the case when the anti-IFN-γ CLAb and anti-TNF-α CLAb are used to capture the secreted proteins IFN-γ and TNF-α, then two FLAbs respectively specific to the IFN-γ and TNF-α are mixed with the product of step (d), in which the two FLAbs respectively carry different fluorophores (for example, FITC and APC, or FITC and PE); alternatively, in the case when the anti-IFN-γ CLAb, anti-TNF-α CLAb and anti-IL-16 CLAb are used to capture the secreted proteins IFN-γ, TNF-α and IL-16, then three FLAbs respectively specific to the IFN-γ, TNF-α and IL-16 are mixed with the product of step (d), in which each of the CLAbs carries a fluorophore different from one another (for example, FITC, APC and PE); and (iii) Each fluorophores of the FLAbs are irradiated/excited and detected by lights at corresponding wavelengths in step (f) and (g); for example, the FITC is preferably excited by a light at the wavelength of 488 nm, thereby producing an emission that can be detected at the wavelength of 525 nm; the APC is preferably excited by a light at the wavelength of 651 nm, thereby producing an emission that can be detected at the wavelength of 660 nm; and PE is preferably excited by a light at the wavelength of 565 nm, thereby producing an emission that can be detected at the wavelength of 574 nm.

Additionally or alternatively, in addition to the secreted protein(s), the method is useful in simultaneously measuring the level of at least one surface protein of the cell. In this case, the solution of step (e) further comprises a FLAb (i.e., a second FLAb) specific to the surface protein. As described above, the second FLAb carries a fluorophore different from the fluorophores of the first FLAb; thus, the first and second FLAbs are respectively excited and detected at different wavelengths. For example, the first and second FLAbs may be respectively conjugated with a FITC molecule and a PE molecule; in this case, the first FLAb used to quantify the secreted protein may be excited by a light at the wavelength of 488 nm thereby producing an emission that can be detected at the wavelength of 525 nm, while the second FLAb used to quantify the surface protein may be excited by a light at the wavelength of 565 nm thereby producing an emission that can be detected at the wavelength of 574 nm.

The present method is characterized in that each water-in-oil droplet of step (b) encapsulates only one cell therein; accordingly, the proteins secreted by the single cell during the incubation time (step (c)) are enclosed in the water-in-oil droplet until the water-in-oil droplet is broken upon the addition of the de-emulsifier (step (d)). The encapsulation of single cell in the water-in-oil droplet ensures that the protein secreted by a single cell can be efficiently captured by the CLAb, and then accurately measured/quantified via the reporter molecule (i.e., the fluorophore of the FLAb) without being mixed/contaminated by the protein secreted from other cells. Thus, the present method could measure the protein levels of individual cells in a population of cells (e.g., a biological sample), in which each cell of the population of cells is encapsulated in one water-in-oil droplet in step (b).

According to some embodiments of the present disclosure, the present method may simultaneously detect the secreted protein(s) and/or surface protein(s) of single cells at high throughput (about 10³ cell/per second); thus, the present method is a potential means to,
(a) distinguish different cell types/subtypes, thereby providing a new insight into clinical treatment and fundamental research; and
(b) discover rare and specific cluster, provide an objective bio-index reflecting the physiological immune status and disease progress, representing a major advancement for precision medicine.

Compared to conventional methods, the present method is advantageous in possessing the following aspects,
(I) CLAbs are used to measure cell secretions outside cells instead of inside cells, and the cells are kept alive throughout the process; and
(II) no extra cell-surface receptors are blocked, allowing the cells to function without being disturbed by receptor inactivation.

Also disclosed herein is a method of making a diagnosis of a disease in a subject. The method comprises,
(a) isolating a biological sample from the subject;
(b) measuring the level of a protein in the biological sample by the present method as described above;
(c) comparing the level of the protein measured in step (b) with that in a control sample derived from a healthy subject; and
(d) making the diagnosis of the disease based on the result of step (c), wherein the difference (i.e., higher or lower) in the respective levels of the protein in the biological sample and control sample is an indication that the subject has the disease.

According to some embodiments, the disease is a cancer; for example, a gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck carcinoma. In one exemplary embodiment, the cancer is head and neck carcinoma.

Examples of the subject suitable for use in the present method include, but are not limited to, a human, mouse, rat, hamster, guinea pig, rabbit, dog, cat, cow, goat, sheep, monkey, or horse. According to preferred embodiments, the subject is a human.

Depending on intended purpose, the biological sample of step (a) may be a whole blood sample, PBMC, serum sample, plasma sample, biopsy sample, urine sample, or mucosa sample. According to some exemplary embodiments, the biological sample is immune cells, for example, monocytes, neutrophils, dendritic cells (DCs), macrophages, T cells, or B cells. According to embodiments of the present disclosure, the protein measured by the present method may be a cytokine or chemokine, such as IFN-α, IFN-β, IFN-γ, TNF-α, TGF-β, IL-1, IL-2, IL-4, IL-5, IL-6, IL-7, IL-8, IL-10, IL-13, IL-16, IL-17, IL-18, IL-23, MCP-1 (also known as “CCL2”), C-X-C chemokine receptor (CXCR) 1, CXCR2, CXCR3, CXCR4, C-X-C chemokine ligand (CXCL) 9, CXCL10, CXCL11, CXCL12, C-C chemokine receptor (CCR) 2, CCR4, CCR5, CCR6, CCR7, or a combination thereof.

In certain embodiments of the present disclosure, the cancer is NPC, and the biological sample is PBMC, in which the protein measured by the present method is IFN-γ, TNF-α, TGF-β, IL-1, IL-4, IL-5, IL-6, IL-8, IL-10, IL-13, IL-16, IL-17, IL-23, MCP-1, or a combination thereof. According to one specific embodiment of the present disclosure, the respective levels of the IFN-7 and TNF-α in the biological sample are higher than those in the control sample, which indicates that the subject has the NPC.

Based on the diagnosis rendered above, a suitable treatment (e.g., an anti-cancer treatment) may be timely administered to the subject (e.g., a patient suffering from the cancer). According to certain embodiments, the disease is a cancer. In the embodiment, in the case when the respective levels of the IFN-γ and TNF-α in the biological sample of the subject are higher than those in the control sample, then an anti-cancer treatment (e.g., surgery, chemotherapy, radiation therapy, immunotherapy, hormone therapy, anti-angiogenic therapy, or a combination thereof) is administered to the subject.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example

### Materials and Methods

### CLAbs.

Two main steps were included in the present study to synthesize CLAbs. First, antibodies and a crosslinker (dibenzocyclooctyne-sulfo-N-hydroxysuccinimidyl ester (DBCO-Sulfo-NHS) were mixed in Dulbecco's phosphate-buffered saline (DPBS) at a molar ratio of 1:10, forming DBCO-modified antibodies. Notably, based on the target cytokines to be captured (e.g., IFN-γ, TNF-α and IL-16), corresponding antibodies were used. The mixture was shaken at room temperature (about 24° C.) for 2 hours. Any unreacted molecules were removed through a 100-kDa spin filter. Second, the purified DBCO-modified antibodies were conjugated to cholesterol-conjugated DNA linkers, each of which comprised a DNA linker, a cholesterol molecule linked to its 5′ end, an azide group linked to its 3′ end, at a molar ratio of 1:15, and the mixture was kept at 4° C. overnight. The nucleotide sequences of the DNA linkers are summarized in Table 1. The CLAbs were purified with a 100-kDa spin filter. The molecular weight (MW) of the synthesized CLAbs was measured by sodium dodecyl-sulfate polyacrylamide gel electrophoresis (SDS-PAGE) with coomassie brilliant blue staining. A pre-stained color protein ladder was used as a reference for MW. Moreover, a protein detection kit was used to determine the CLAb concentration, following the standard protocol.

TABLE 1
Nucleotide sequences of DNA linkers for linking
DBCO-modified antibody and cholesterol molecule

| DNA linker | Nucleotide sequence | SEQ ID NO |
|---|---|---|
| 1 | Chol-TTTTTTTCACTCATTCAA- | 1 |
| | TACCCTACGTCTACCCTAC-Azide | |

### Microfluidic Experimental Setup

The single cells were encapsulated in the water-in-oil droplets by a microfluidic droplet generator, which included one inlet for the continuous oil phase and two inlets for the aqueous phases for the simultaneous loading of cells and stimulation reagents. The device was operated by introducing cells (3×10⁶ cells/mL) and a chemical solution for cell stimulation (e.g., 100 ng/mL PMA, 2 μg/mL ionomycin, 2.5 μg/mL Con A, or 2.5 μg/mL PHA-L) into two inlets at a flow rate of 300 μL/hr. The carrier oil consisting of 0.2% v/v surfactant (PICO-SURF®, a biocompatible surfactant) dissolved in NOVEC™ 7500 (a nonflammable solvent), a carrier oil widely used in a variety of micro-droplet and pico-droplet applications, was used at a flow rate of 600 μL/hr. The flow rates of both the aqueous phases and the carrier oil were controlled by syringe pumps to produce uniform water-in-oil droplets with a diameter of approximately 34 μm.

### CLAb Attachment on the Cell Membrane

A sample containing 1×10⁶ cells was concentrated via centrifugation at 1,000 rpm for 5 minutes and re-dispersed in 100 μL of DPBS. Then, the solution thus produced was incubated with 1 μg of CLAbs for 30 minutes to allow the CLAbs to spontaneously anchor to the cell surface. Finally, the CLAb-grafted cells were washed with DPBS and re-suspended in cell culture medium.

### Capturing Cytokines

CLAb-grafted cells treated with/without activators (e.g., PMA, ionomycin, Con A, or PHA-L) were incubated in a culture medium at 37° C. for 6 hours for cytokine production. Target cytokines were captured on the cell surface via the CLAbs.

For the single-cell assay, single cells were incubated with stimuli in droplets under the same incubation conditions to capture the cell secretions. After that, the droplets were placed at 4° C. to stop the cell secreting, and then were de-emulsified to recover the cells by adding a de-emulsifier (1H,1H,2H,2H-perfluoro-1-octanol; a fluorotelomer alcohol) into the oil at a 1:5 ratio. The extracted cells were re-suspended in DPBS buffer for further usage.

### Labelling and Screening

For samples with captured secretions to be stained by fluorophore-tagged antibodies and analyzed by flow cytometry, cells were washed with 1 mL of cold DPBS and re-suspended in 100 L of cold DPBS. One microgram of each fluorophore-tagged antibody was added to the solution to label the secretions, and the cells were incubated at 4° C. for 1 hour. Notably, cell receptors were stained simultaneously if needed. Any unbound fluorophore-tagged antibodies were then washed out in two washes with 1 mL of cold DPBS, followed by resuspension of the cells in 500 μL of cold DPBS. The cells with fluorescence signals were then screened by flow cytometry.

### Data Analysis

All samples were analyzed in triplicate, and the standard deviation was plotted as the error bar. The statistical significance level of results was evaluated using P-values obtained from two-tailed unpaired student's t-tests.

### Example 1 Characterization of CLAbs

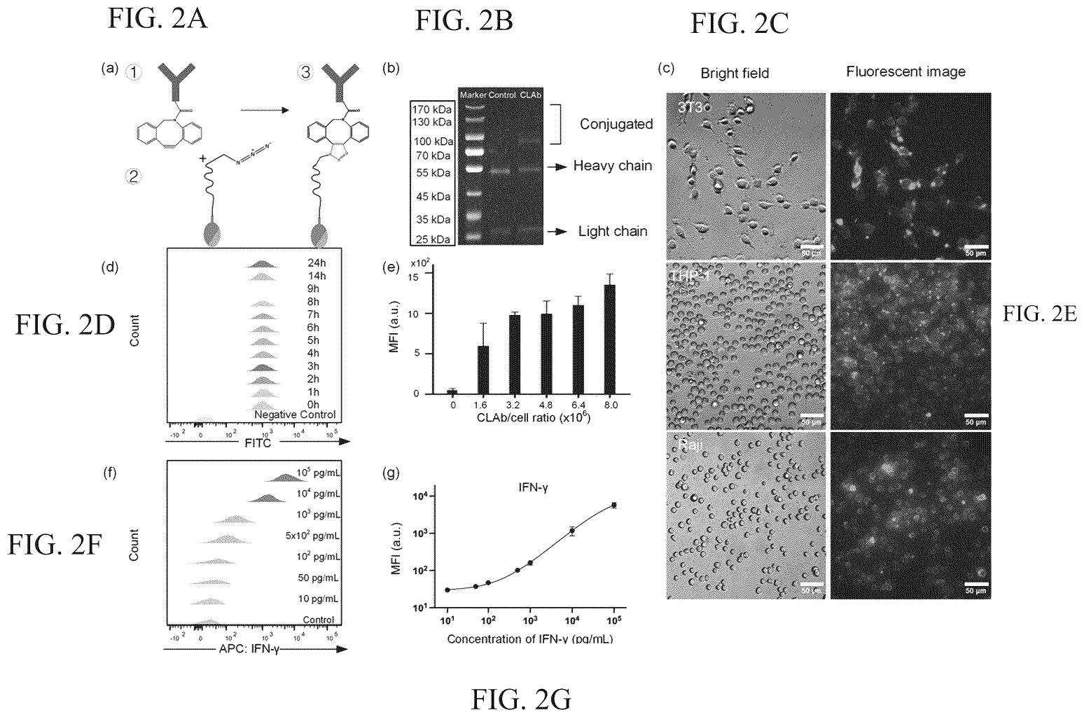

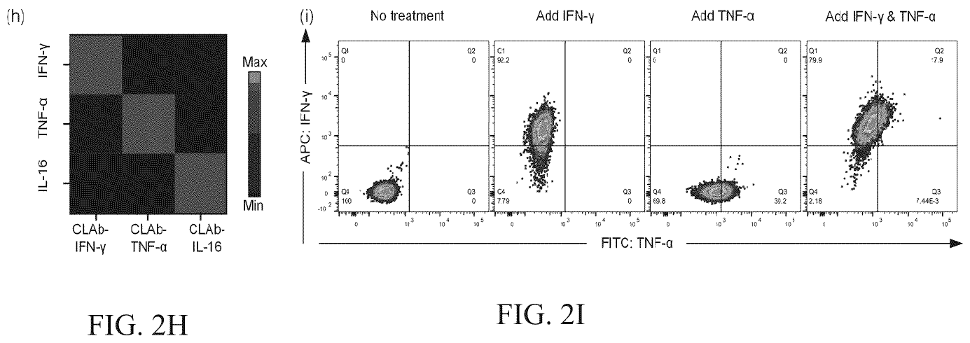

CLAbs were synthesized via the copper-free click chemistry method described in the “Materials and Methods” section. The DBCO-sulfo-NHS ester reacted with the exposed amine side chains of antibodies to produce DBCO antibodies. The cholesterol-conjugated DNA linkers were then covalently linked to the DBCO-modified antibodies through an alkyne-azide cycloaddition between the DBCO and azide groups, resulting in the formation of CLAbs (FIG. 2a). Different CLAbs that enabled the capture of target cytokines (for example, IFN-γ, TNF-α and IL-16) were prepared by conjugating appropriate antibodies. SDS-PAGE was performed to measure the MW changes after the click chemistry reaction. For unreacted antibodies, two series of bands were observed in the lane for control antibodies: upper bands (MW: about 55 kDa, heavy chains) and bottom bands (MW: about 25 kDa, light chains) (FIG. 2b). After the reaction, additional bands appeared in the upper band region (MW: 70 kDa to 130 kDa) in the lane for CLAbs (FIG. 2b), which indicated successful conjugation of about 58.4% of the antibodies. Notably, more than a 15-kDa change (the MW of one cholesterol-conjugated DNA linker is equivalent to 12,900 kDa) in MW and multiple extra bands (about 4) reflected that many DNA strands were conjugated to the same antibodies.

The binding affinity of CLAbs was evaluated by labeling the grafted CLAbs by FITC-conjugated goat anti-mouse IgG (Ex/Em: 488/519 nm) on different cells, including suspension cells (Raji, THP-1) and adherent cells (3T3) (FIG. 2c). The antibodies utilized for the conjugation of CLAbs were all derived from mice and were amenable to labeling with a specific fluorophore-tagged secondary antibody. Almost all cells were grafted with CLAbs and exhibited fluorescence signals (FIG. 2c). Unlike most cholesterol-modified molecules that are easily internalized through endocytosis, CLAbs were stably anchored on the cell surface for 24 hours. To demonstrate the CLAb grafting stability, CLAb-grafted cells were incubated for different time intervals (including 0, 1, 2, 3, 4, 5, 6, 7, 8, 9, 14, or 24 hours), then were labeled with FITC-conjugated goat anti-mouse IgG for about 30 min before being analyzed via flow cytometry. The fluorescence peaks remained mostly unchanged over time, demonstrating the high stability of CLAbs anchored to the cell surface over 24 hours (FIG. 2d). The mean fluorescence intensity (MFI) of the peak fluctuated while approaching a plateau (about 1148 a.u.) in the first 3 hours and then remained stable (about 1067 a.u.) for about 24 hrs (FIG. 2d).

To determine the CLAb anchoring efficiency, cells were grafted with CLAbs by varying the ratio of CLAb to cell (i.e., 1.6×10⁶: 1, 3.2×10⁶: 1, 4.8×10⁶: 1, 6.4×10⁶: 1, and 8×10⁶. 1), the products were subsequently labeled with FITC-conjugated goat anti-mouse IgG for flow cytometry screening. The MFI peak shifted from 596.5±282.14 a.u. to 1355±134.35 a.u. (n=3, mean±s.d.), when the ratio was increased from 1.6×10⁶ 1 to 8×10⁶: 1 (FIG. 2e). In the following experiments, the CLAb to cell ratio was optimized to 3.2×10⁶. 1 to ensure that sufficient CLAbs were anchored on each cell to capture secreted cytokines.

### Example 2 Measurement of Cytokines with the CLAbs-Grafted Cells of Example 1

Calibration curves were generated with known concentrations of recombinant proteins in the bulk solutions. A set of standard protein samples was prepared as a mixture of cytokines in 0.1% bovine serum albumin (BSA)/phosphate buffered saline (PBS) solutions. Before performing the assays, cells were treated with brefeldin A (5 μg/mL) for 6 hours to inhibit the transport of their own cellular secretions to prevent contamination and were then grafted with CLAbs. The CLAb-grafted cells were mixed with standard protein samples for about 1.5 hours to capture the cytokines. After that, the cells with captured cytokines were labeled with fluorophore-tagged antibodies and subsequently screened by flow cytometry. IFN-γ was tested to proof the concept. The flow cytometry results showed a cytokine concentration-dependent increase in fluorescence (FIG. 2f). The MFI increased from 26.53±1.44 a.u. to 5712.33±960.13 a.u. (n=3, mean±s.d.) with the IFN-7 concentration increasing from 0 pg/mL to 10⁵ pg/mL in a dose-dependent manner (FIG. 2g). Different cytokines (TNF-α and IL-16) were tested to validate the assay's flexibility. The detection limits to measure IFN-γ, TNF-α and IL-16 in the bulk solutions were 10³ pg/mL, 10⁴ pg/mL and 10⁷ pg/mL, respectively. The sensitivity and detection limit were mainly dependent on, (1) the number of CLAb molecules on cell surface, and (2) the flow cytometry capability to measure the labelled fluorescence signals. Notably, the number of anchored CLAbs on the cell membrane varied from cell to cell, which might correlate with the cell size and the nature of the cell membrane.

The specificity of CLAbs for target cytokines was assessed with corresponding recombinant proteins. All antibodies used were monoclonal antibodies to minimize cross-reactivity. The data of crosstalk reactivity tests via measuring the target cytokines in solutions containing mixed recombinant IFN-γ (10 ng/mL), TNF-α (10 ng/mL) and IL-16 (100 ng/mL) indicated that crossreactivity was lower than 2% (FIG. 2h).

A multiplexed assay was performed by simultaneously measuring IFN-γ and TNF-α by anchoring multiple CLAbs containing different specific antibodies (i.e., CLAb-IFN-γ and CLAb-TNF-α) on the cell surface. Notably, as the background fluorescence signals of flow cytometry experiments varied under different experimental conditions, in each experimental setting, before measuring the samples, characterizing the negative control group was necessary to determine the gate fluorescence (thresholds). The results of flow cytometry demonstrated that when measuring the IFN-γ, which was captured by the cells with high efficiency (92.2%), as shown by the APC fluorescence signals (FIG. 2i). A similar result was obtained when measuring the TNF-α, in which TNF-α was captured by the cells (30.2%), as shown by the FITC fluorescence signals (FIG. 2i). When the samples containing both IFN-γ and TNF-α were measured, the two secreted proteins were simultaneously captured by the cells (17.9%), as shown by the dual fluorescence signals (FIG. 2i). Further, the cell secreted compounds in cellular supernatants were also tested in the example. CLAb-grafted Raji cells were chemically stimulated to capture their own secreted IL-16 in different incubation periods (including 0, 2, 4, 6, and 24 hours). It was found that the fluorescence enhancement gradually plateaued at 24 hours after PMA and ionomycin stimulations.

### Example 3 Single-Cell Multiplexed Analysis

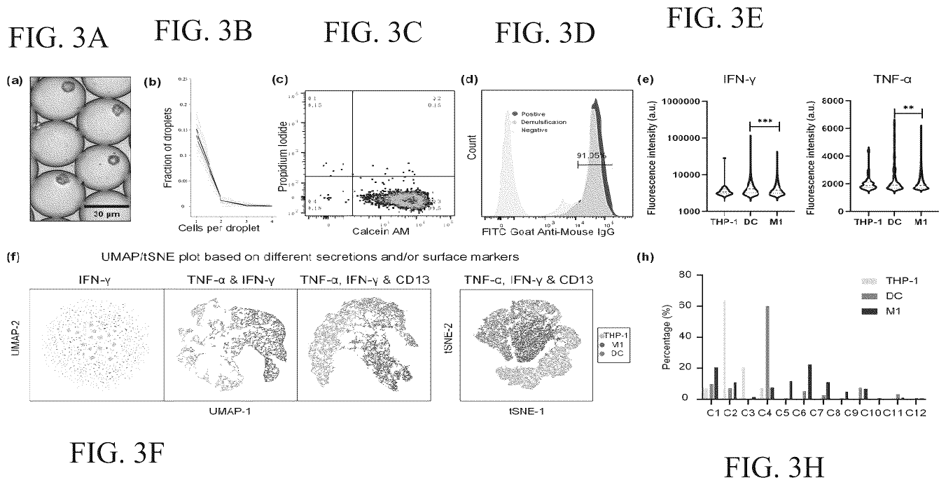

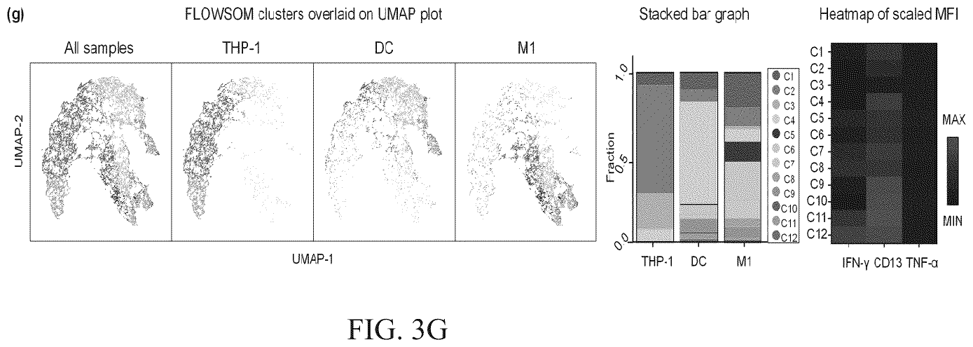

As described above, two steps were included in the present study for single-cell multiplexed phenotyping. In step 1 (the capturing step), single cells grafted with CLAbs were encapsulated in picoliter water-in-oil droplets (average: 20.57 pl, SEM: 1.76 pl, n=25; FIG. 3a) with an encapsulation efficiency of approximately 15.18% single cell-containing droplets (FIG. 3b). CLAb-grafted single cells were incubated within the droplets for 6 hours to capture their own secreted cytokines on the cell surface. In step 2 (the labeling/screening step), the droplets were then broken to re-suspend the cells with captured cytokines in a bulk solution. The fluorophore-tagged antibodies were subsequently uploaded to label the captured cytokines and surface proteins for subsequent detection. The single cells labeled with fluorescence tags were then screened by flow cytometry to simultaneously measure their secretions and surface proteins with a throughput of about 10³ cells per second. Notably, the cell viability after extraction from the droplets through chemical de-emulsification was verified using live/dead stains, and the staining results demonstrated a high cell survival rate (about 99.5%) measured through flow cytometry (FIG. 3c). Moreover, after de-emulsification, CLAb attachment was assessed with FITC-conjugated goat antimouse IgG, and the staining results indicated that most CLAbs remained on cells (about 91.05%), which emitted fluorescence signals (FIG. 3d).

Multiple cytokines (IFN-γ and TNF-α) and a surface protein (CD13) were simultaneously assayed to identify monocytes (THP-1) and derived cells (M1 and DCs). To analyze variation in the secretions (IFN-γ and TNF-α) among the three cell types, an Ordinary one-way ANOVA test with post-hoc Tukey HSD (95% confidence) was performed using data from 10⁴ single cells randomly selected from each cell type (FIG. 3e). Different IFN-γ amounts secreted by THP-1, DCs and M1 were observed, while a high level of cell heterogeneity was observed in M1 cells (FIG. 3e). To visualize patterns of clustering based on different expression levels of cytokines and/or surface proteins, dimension reduction algorithms such as UMAP were conducted. Three cell types (THP-1, M1 and DC) were clearly distinguished by the multiplexed assay of secretions and surface proteins. Notably, by measuring only cell surface protein (CD13), THP-1, M1 and DC could not be well identified. On the other hand, by assaying only one type of secreted cytokines (IFN-γ or TNF-α), the challenge of differentiating M1 and DC remained, despite significant differences in expression levels. Indeed, to distinguish three different cell types well, it was necessary to simultaneously measure multiple phenotypes including surface protein (CD13) and secretions (IFN-γ and TNF-α). A 2D plot constructed by the UMAP algorithm was conducted to visualize three cell types classified with clear boundaries (FIG. 3f).

A combination of an unsupervised clustering algorithm (FlowSOM) and UMAP was utilized to generate more specific clusters that exhibit distinct expression levels for each marker. The identified clusters can be determined by heatmaps that display the scaled total MFI of each marker (IFN-γ, TNF-α, and CD13), providing insight into the expression patterns of different markers. Overlaying the clusters on a UMAP plot can reveal differences in the distribution of the clusters among samples, which was further examined by stacked bar graphs that depict the proportional representation of each cluster in three cell lines. Based on the expression levels of IFN-7, TNF-α, and CD13, the cells were classified into 12 clusters with different expression levels displayed in a heatmap (FIG. 3g). CD13, a transmembrane metalloprotease, was widely expressed in all cell clusters (FIG. 3g). THP-1 cells (92.37%) were classified into clusters 1-3 (C1-3; FIG. 3g), indicating low cytokine expression levels in general. DCs (80.91%) were mainly classified into four clusters (C1, C4, C9 and C11; FIG. 3g). A total of 15.3% of the DCs were classified into 5 clusters (C7-9, C11-12; FIG. 3g), indicating high levels of IFN-γ expression. M1 macrophages were predominantly classified into multiple clusters (C1-2, C4-9; FIG. 3g), revealing their innate cell heterogeneity. A total of 22.93% of the M1 macrophages were classified in cluster 5 and cluster 7, indicating high expression levels of cytokines (IFN-γ and TNF-α) and surface proteins (CD13) (FIG. 3h).

### Example 4 Clinical Analysis

CLAb technology was applied to profile cytokine secretion and surface markers in human PBMCs from one healthy donor to compare the activation results of different chemical stimulations in vitro. Here, a 6-parameter panel was designed to characterize the secretion ability of T cells and B cells stimulated by three kinds of chemicals (including, PMA plus ionomycin; Con A; and PHA-L), and basal groups with unstimulated PBMCs from the same donor were used as controls. The human PBMCs from one healthy donor were recovered in complete RPMI-1640 culture medium for 24 hours after thawing and grafted with CLAbs. The single cells were then co-encapsulated with/without stimulation in droplets to capture produced secretions on the cell surface with the CLAbs, followed by labeling and screening steps. A common protocol was used to stain the surface proteins (CD3, CD4, CD8 and CD19) of PBMC at 4° C. for 1 hour. To analyze the results of flow cytometry, the live lymphocytes were gated, and a Downsample plugin was employed for normalization to obtain 3,000 single-cell datapoints for each physiological condition (i.e., basal treatment; PMA plus ionomycin treatments; Con A treatment; or PHA-L treatment), enabling statistical analysis.

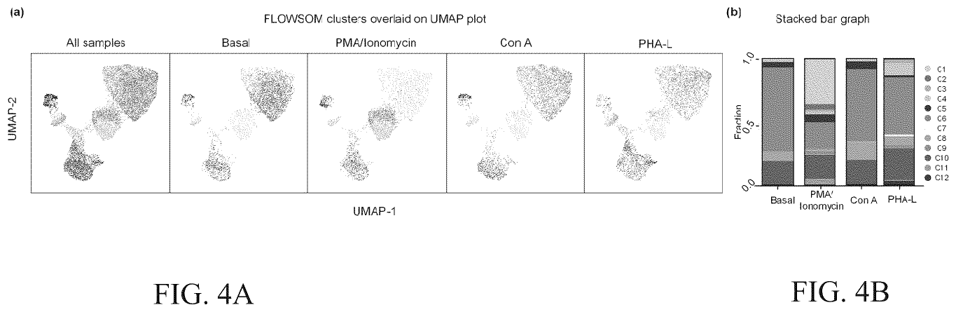

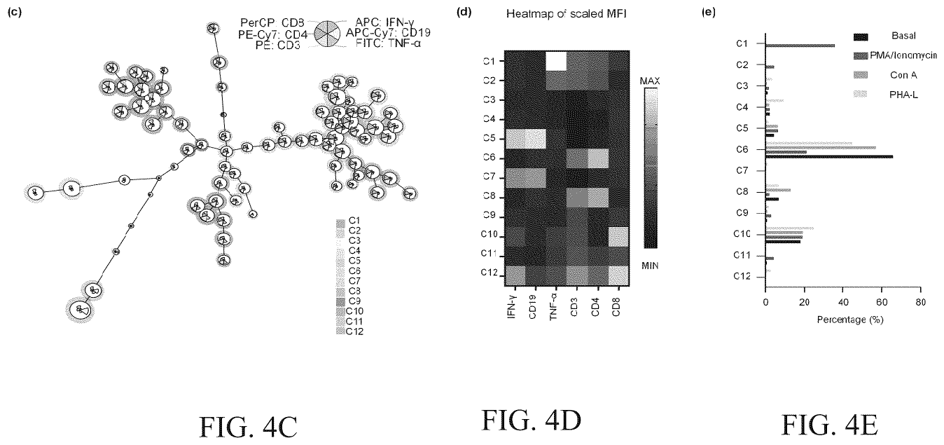

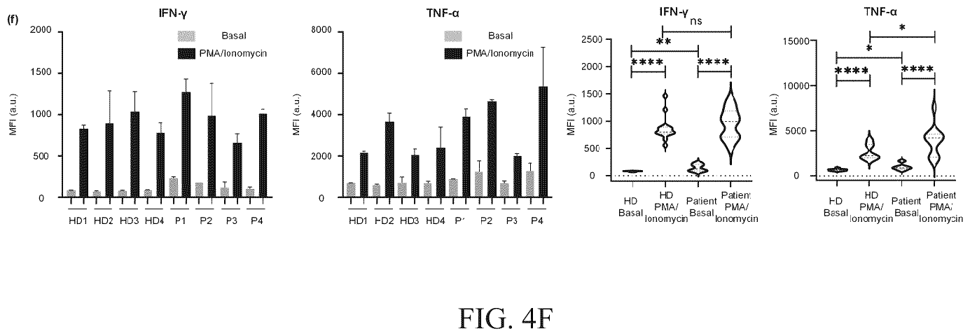

The high-dimensional single-cell data were mapped onto the UMAP plot with 12 clusters calculated by the clustering algorithm FlowSOM to visualize the functional phenotypes under different stimulations. The heatmap depicted the relative expression of IFN-γ and TNF-α in each cluster, thus explaining cluster identity. Notably, all T lymphocytes express CD3, while helper T cells express CD4 and cytotoxic T cells express CD8. B lymphocytes, which are important in adaptive immune responses, could be recognized by CD19 expression. Moreover, CD3-CD19-cells are usually natural killer (NK) cells and DCs from the innate immune system. To analyze how PMA/ionomycin (Con A and PHA-L) altered the cell functions in different cell clusters, samples with various treatments were de-barcoded in UMAP plots. The population distribution changes in the cell clusters under different stimuli indicated the effects of treatments (FIG. 4a). According to previous research, PMA/ionomycin stimulation would result in changes in cytokine secretion levels, reflecting the effects of xenobiotics on immunotoxicity assessment.

The fractions of different cell clusters were depicted in a stacked chart (FIG. 4b). A FlowSOM tree was constructed to display all clusters with marker expression. Here, the C1 group consisted of CD3⁺CD4⁺ T cells exhibiting high level of TNF-α secretion, whereas the C2 group consisted of CD3⁺CD4⁺ T cells with relatively less TNF-α secretion. The C3 and C4 groups included quiescent lymphocytes that did not secrete any cytokines. The C5 and C7 groups contained CD19⁺IFN-γ⁺ B cells, with C5 also secreting few TNF-α. The C6 and C8 groups consisted of quiescent CD3⁺CD4⁺ T cells without any secretions. The C9 and C11 groups included CD3⁺CD4⁻CD8⁻ T cells that did not secrete any cytokines. The C10 group contained quiescent CD3⁺CD8⁺ T cells, while the C12 group consisted of CD3⁺CD8⁺ T cells that exhibited high secretion levels of both IFN-γ and TNF-α. To focus on functional cells, the cells were divided into three main subpopulations based on the secretion levels, including (1) a cell subpopulation (CS1) that produced no cytokine (C3-4, C6 and C8-11), (2) a cell subpopulation (CS2) that only secreted IFN-γ (C7) or TNF-α (C1-2), and (3) a cell population (CS3) that was bifunctionally activated to produce IFN-7 and TNF-α (C5 and C12) (FIG. 4c).

In the unstimulated group (basal), the CS1 cell subpopulation (quiescent cells) represented 95.1% of cells, while a cluster of CD19⁺ B cells (C5, 4.6%) that secreted IFN-γ and TNF-α emerged in relation to the active immune responses (FIG. 4d). Under PMA/ionomycin stimulation, the CS2 cell subpopulation increased to 40.46% (FIG. 4e). Specifically, large amounts of the cells (C1-2) that actively secreted TNF-α were observed (FIG. 4e), indicating enhanced proinflammatory or immunosuppressive effects. Moreover, the TNF-α cell population in a PMA- and ionomycin-stimulated sample was replenished from cells originating in quiescent CD4⁺ T cells (C6 and C8) in the basal state (FIG. 4e). Under Con A stimulation, the CS3 cell subpopulation was increased and a population of CD19* B cells (C5) that actively secreted both IFN-γ and TNF-α emerged (6.43%; FIG. 4e). On the other hand, under PHA-L stimulation, a distinctive cell cluster (C12) exhibiting elevated levels of IFN-γ and TNF-α in CD3⁺CD8⁺ T cells emerged, suggesting heightened cytotoxic activities in adaptive T-cell immune responses (FIG. 4e). In general, PMA/ionomycin stimulation elicited a stronger immune response than Con A or PHA-L stimulation, generating a larger CS2/CS3 cell subpopulation (47.14% under PMA/ionomycin stimulation vs. 7.42% under Con A stimulation and 6.62% under PHA-L stimulation).

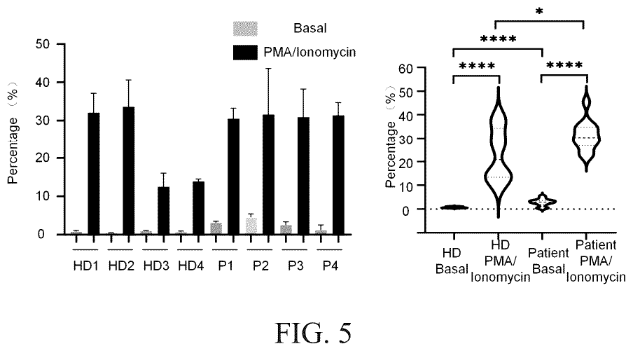

To assess the potential applications of this platform in clinical use, the PBMC samples from four healthy donors and four nasopharyngeal carcinoma (NPC) patients with different cancer stages were analyzed. The secretion of IFN-γ and TNF-α was detected by CLAbs to compare differences between patients and healthy donors. The positive control involved activating the cells with the PMA/ionomycin solution. The secretion of IFN-γ and TNF-α significantly increased under PMA/ionomycin stimulation, regardless of whether the donors were healthy or NPC patients (FIG. 4f). The average quantity of IFN-γ secreted by NPC patients was comparable to that secreted by healthy donors (N=4 patients/healthy donors, n=3; NPC patients: 954.1±276.1 a.u.; healthy donors: 871.3±232.1 a.u., mean±s.d., P=0.443; FIG. 4f), while NPC patients displayed more TNF-α secretion than healthy donors after activation (N=4, n=3; NPC patients: 3923±1692 a.u.; healthy donors: 2662±845.4 a.u., mean±s.d., P=0.028; FIG. 4f). However, in the case of the basal state, two-tailed t-test (unpaired, 95% confidence) analysis indicated that PBMCs taken from NPC patients exhibited a higher secretion level of TNF-α than those obtained from healthy donors (N=4, n=3; NPC patients: 1013±372.2 a.u.; healthy donors: 663.7±114.6 a.u., mean±s.d., P=0.011; FIG. 4f) after 6-hour incubation. A similar result was observed for basal IFN-γ secretion, with the fluorescence intensities of NPC patient samples being approximately twofold higher than those from healthy donors (N=4, n=3, NPC patients 145.9±65.48 a.u.; healthy donors 81.52±7.13 a.u., mean±s.d., P=0.007; FIG. 4f). Moreover, compared with the healthy donor samples, NPC patient samples contained approximately four times as many cells exhibited active IFN-γ/TNF-α-secretion without stimulations (N=4, n=3; NPC patients: 2.82±1.48%; healthy donors: 0.70±0.29%, mean±s.d., P<0.0001, two-tailed unpaired t-test; FIG. 5). The ratio of active immune cells in NPC patients suggested a potential bio-index reflecting the physiological immune status and disease progress.

In conclusion, the present disclosure provides a microfluidic cell membrane immunosorbent assay using CLAbs. According to the examples of the present disclosure, this assay was capable of simultaneously measuring multiple cytokines and surface proteins of an individual cell to identify extended biomarkers for diagnosis. Notably, CLAbs were used to measure single-cell secretions to determine cell physiological states, and the cells were kept alive. Moreover, no extra cell-surface receptors were blocked, allowing the cells to function without disturbance by receptor inactivation. As a proof-of-concept, multiple surface proteins (CD3, CD4, CD8 and CD19) and secreted cytokines (IFN-γ and TNF-α) were simultaneously measured for single cells at a throughput of about 10³ cells per second. Together, the cell type (surface proteins) and functions (cytokines) of single cells offered novel insights into the physiological consequences of molecular heterogeneity in patients' immune systems. The multiplexed phenotyping of THP-1 cells, M1 macrophages and DCs allowed profiling to estimate the efficiency of cellular differentiation. Moreover, PBMC samples were analyzed, in which compared with healthy donor samples, NPC patient samples contained a higher percentage of cytokine (IFN-γ and TNF-α)-secreting cells with significant diversity, closely related to the disease conditions. With the advance of high-throughput single-cell screening, crucial single cells, with active cytokine secretions and relevant surface proteins could be rapidly identified and counted as a potential bio-index to determine the disease progress with reduced time and cost.

It will be understood that the above description of embodiments is given by way of example only and that various modifications may be made by those with ordinary skill in the art. The above specification provides a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of measuring the level of at least one protein of a cell comprising:
(a) mixing the cell with a cholesterol-linked antibody (CLAb) to produce a CLAb-conjugated cell, wherein the CLAb comprises a first antibody specific to a first protein secreted from the cell, and a cholesterol linked to the first antibody;
(b) encapsulating the CLAb-conjugated cell of step (a) in a water-in-oil droplet;
(c) incubating the product of step (b) at a temperature for a period of time;
(d) adding a de-emulsifier to the product of step (c);
(e) mixing the product of step (d) with a solution comprising a first fluorophore-linked antibody (FLAb), wherein the first FLAb comprises a second antibody specific to the first secreted protein, and a first fluorophore linked to the second antibody;
(f) irradiating the product of step (e) with a light having a first wavelength to excite the first fluorophore; and
(g) detecting the intensity of a first signal emitted by the excited first fluorophore of step (f) at a second wavelength, in which the first signal represents the measurement of the level of the first protein.

2. The method of claim 1, wherein in step (c), the product of step (b) is incubated at 37° C. for at least 4 hours.

3. The method of claim 1, wherein the solution of step (e) further comprises a second FLAb, which comprises a third antibody specific to a second protein expressed on the surface of the cell, and a second fluorophore linked to the third antibody, wherein the first and second fluorophores are different.

4. The method of claim 3, further comprising,
(f-1) irradiating the product of step (e) with a light having a third wavelength to excite the second fluorophore before or concurrent with step (f); and
(g-1) detecting the intensity of a second signal emitted by the excited second fluorophore of step (f-1) at a fourth wavelength, in which the second signal represents the measurement of the level of the second protein.

5. The method of claim 1, wherein in step (b), the CLAb-conjugated cell of step (a) is encapsulated with a cell stimulant in the water-in-oil droplet.

6. The method of claim 5, wherein the cell is an immune cell, and the cell stimulant is concanavalin A (Con A), phorbol myristate acetate (PMA), phytohaemagglutinin (PHA), ionomycin (Iono), lipopolysaccharide (LPS), tetanus toxoid (TT), or a combination thereof.

7. A method of treating a cancer in a subject, comprising,
(a) isolating a biological sample from the subject;
(b) measuring the level of the at least one protein in the biological sample of step (a) by the method of claim 1;
(c) comparing the level of the at least one protein measured in step (b) with that in a control sample derived from a healthy subject; and
(d) subjecting the subject to an anti-cancer treatment based on the result of step (c), wherein the level of the at least one protein in the biological sample of the subject is higher or lower than that in the control sample.

8. The method of claim 7, wherein the anti-cancer treatment is surgery, chemotherapy, radiation therapy, immunotherapy, hormone therapy, anti-angiogenic therapy, or a combination thereof.

9. The method of claim 7, wherein the biological sample is a peripheral blood mononuclear cell (PBMC) sample; and the protein is interferon-gamma (IFN-γ), tumor necrosis factor-alpha (TNF-α), tumor growth factor-beta (TGF-β), interleukin (IL)-1, IL-4, IL-5, IL-6, IL-8, IL-10, IL-13, IL-16, IL-17, IL-23, monocyte chemoattractant protein-1 (MCP-1), or a combination thereof.

10. The method of claim 9, wherein the cancer is nasopharyngeal carcinoma (NPC), and the level of the IFN-γ and TNF-α in the biological sample of the subject is higher than that in the control sample.

11. The method of claim 7, wherein the subject is a human.
